(19) United States
(12) Patent Application Publication
(10) Pub. No.: US 2023/0293823 A1
(43) Pub. Date: Sep. 21, 2023

(54) Medicine Dosage Control Injecting Device

(71) Applicants: Georganne Erickson, Cave Creek, AZ (US); Shelly Felton, Cypress, TX (US)

(72) Inventors: Georganne Erickson, Cave Creek, AZ (US); Shelly Felton, Cypress, TX (US)

(21) Appl. No.: 17/697,012

(22) Filed: Mar. 17, 2022

(51) Int. Cl. A61M 5/315

(52) U.S. Cl. CPC A61M 5/31553; A61M 5/31585; A61M 2205/583; A61M 2005/3126

(57) ABSTRACT

A medicine dosage control injecting device for adjusting to an accurate dosage of medicine to be injected includes a dispenser being portable and having a pen shaped body. The dispenser contains a cartridge wherein the cartridge stores medicine. The dispenser has a rotational sensor and a cartridge sensor. A dosage ring is coupled to an end of the dispenser and can rotate laterally. The dosage ring is in electrical communication with the rotational sensor. The dosage ring is configured for rotating to adjust a dosage within the cartridge. A display is in electric communication with the rotational sensor and the cartridge sensor. The display shows the numerical amount of the dosage within the cartridge. An injection button is coupled to the end of the dispenser. The injection button is pushed in towards the dispenser wherein the cartridge releases the medicine through an injection port.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to liquid medicine injectors and more particularly pertains to a new liquid medicine injector for adjusting to an accurate dosage of medicine.

### (2) Description of Related Art Including Information Disclosed Under 37 CFR 1.97 and 1.98

The prior art relates to liquid medicine injectors. The prior art includes a variety of liquid medicine injectors containing cartridges to store the medicine in. Furthermore, the prior art includes a variety of liquid medicine injectors having a pen shape body. Known prior art lacks a liquid medicine injector having a pen body comprising a cartridge with an adjustment ring to accurately store and deliver the correct dosage.

### BRIEF SUMMARY OF THE INVENTION

An embodiment of the disclosure meets the needs presented above by generally comprising a dispenser being portable and having a pen shaped body. The dispenser contains a cartridge wherein the cartridge stores medicine. The dispenser has a rotational sensor and a cartridge sensor. A dosage ring is coupled to an end of the dispenser and can rotate laterally. The dosage ring is in electrical communication with the rotational sensor. The dosage ring is configured for laterally rotating to adjust a dosage within the cartridge. A display is in communication with the rotational sensor and the cartridge sensor. The display shows the numerical amount of the dosage within the cartridge from a value provided by the rotational sensor and the cartridge sensor. An injection button is coupled to the end of the dispenser. The injection button is configured for a force to push in the injection button towards the dispenser wherein the cartridge releases the medicine through an injection port.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a medicine dosage control injecting device according to an embodiment of the disclosure.

FIG. 2 is a rear view of an embodiment of the disclosure.

FIG. 3 is a left side view of an embodiment of the disclosure.

FIG. 4 is a right side view of an embodiment of the disclosure.

FIG. 5 is a detail view of an embodiment of the disclosure.

### DETAILED DESCRIPTION OF THE INVENTION

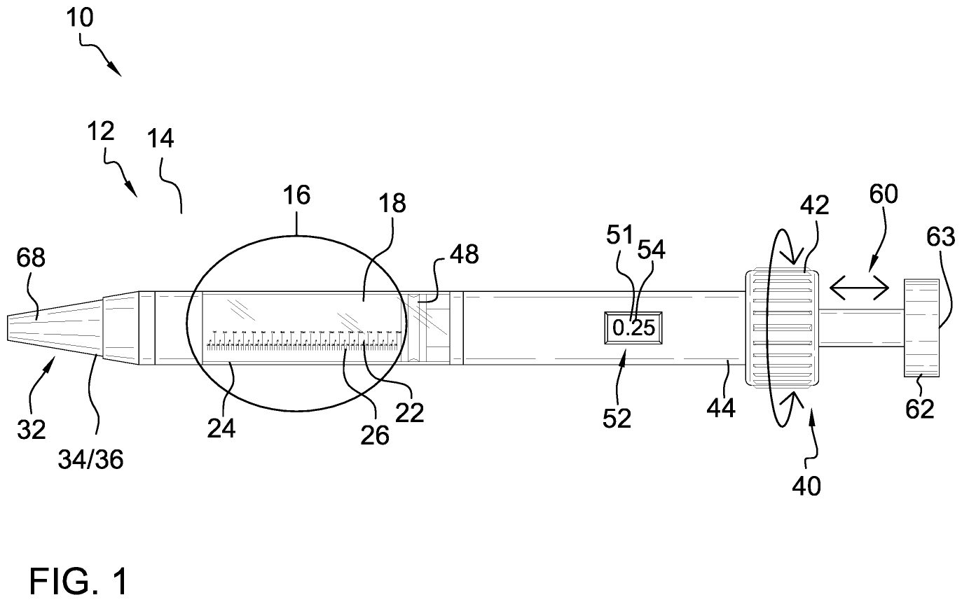

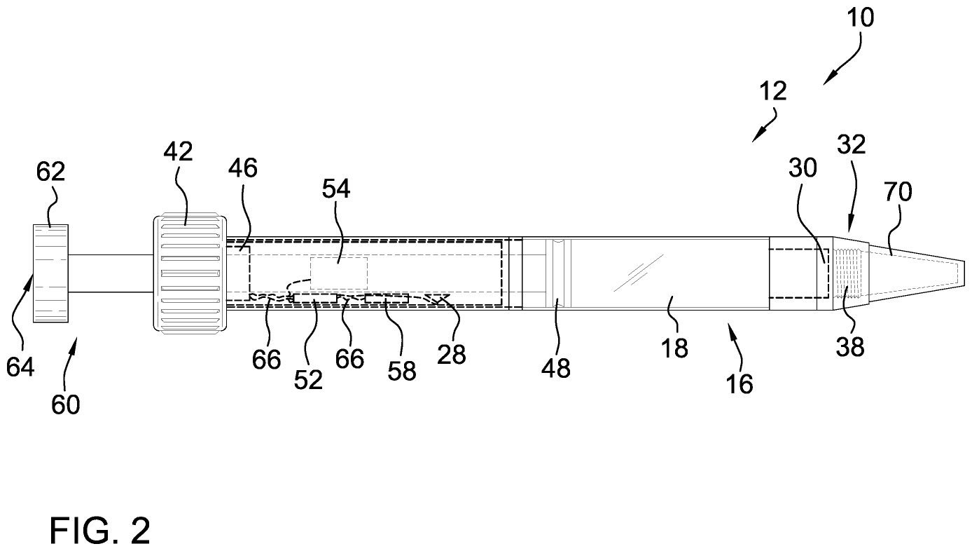

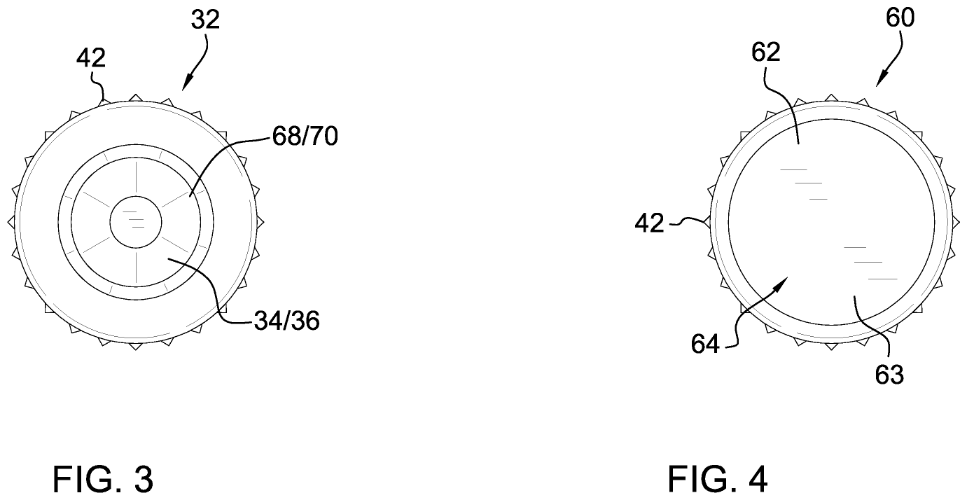

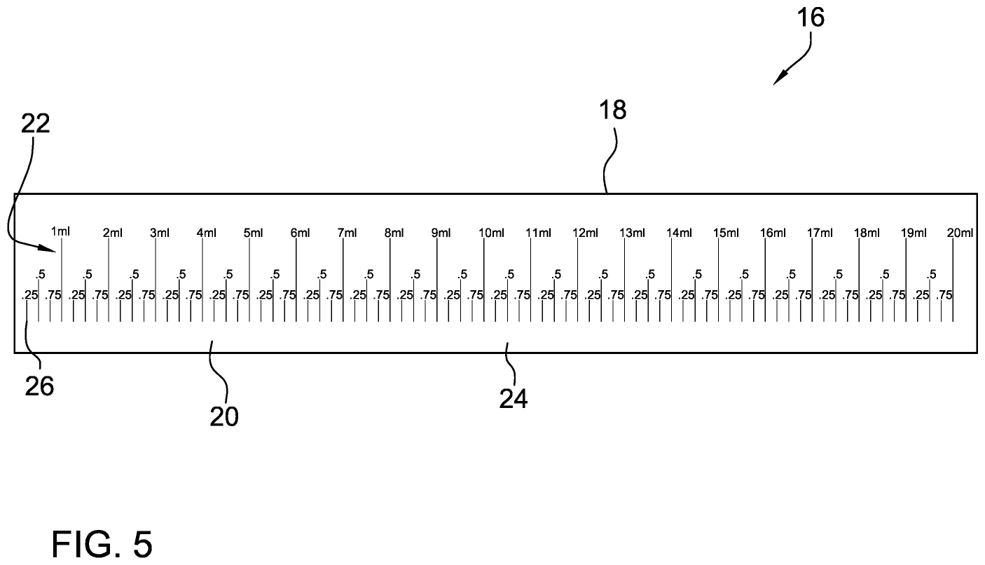

With reference now to the drawings, and in particular to FIGS. 1 through 5 thereof, a new liquid medicine injectors embodying the principles and concepts of an embodiment of the disclosure and generally designated by the reference numeral 10 will be described.

As best illustrated in FIGS. 1 through 5, the medicine dosage control injecting device 10 generally comprises a dispenser 12. The dispenser 12 has a pen shaped body 14. The dispenser 12 has a cartridge 16. The cartridge 16 has a cylindrical body 18. The purpose of the cartridge 16 is to store a medicine 20 within itself. The cartridge 16 is transparent and has a measuring scale 22 on an exterior surface 24 of the cartridge 16. The measuring scale 22 measures within a ¼ milliliter increments 26. The cartridge 16 is in electric communication with a cartridge sensor 28.

An end 30 of the dispenser 12 is an injection port 32. The injection port 32 is an inlet 34 and an outlet 36 for the medicine 20. The injection port 32 has a threading 38. The injection port 32 can be engaged by threading to a variety of caps. A distal end 40 of the dispenser 12 relative to the injection port 32 is a dosage ring 42.

The dosage ring 42 is coupled to an exterior surface 44 of the dispenser 12. The dosage ring 42 is in electric communication with a rotational sensor 46. A rubber plunger head 48 is mechanically coupled to the dosage ring 42. The rubber plunger head 48 is positioned within the cartridge 16. The dosage ring 42 controls the amount of the medicine 20 within the cartridge 16 wherein the rubber plunger head 48 pushes out or pulls in the medicine 20 relative to a dosage 50 set from the dosage ring 42.

As the dosage ring 42 rotates, the rubber plunger head 48 retracts backward within the cartridge 16. The rubber plunger head 48 creates a vacuum suction within the cartridge 16 to draw in the medicine 20 into the cartridge 16. When the dosage ring 42 rotates in the opposite direction, the rubber plunger head 48 protrudes forward within the cartridge 16 to expel the medicine 20 within the cartridge 16.

A display 52 is coupled to the dispenser 12. The display 52 is a light emitting diode screen 54 and is in electric communication with a micro processor 56 where the micro processor 56 sends a numerical amount 51 of the dosage 50 to the display 52. The micro processor 56 is in electric communication with the rotational sensor 46 and the cartridge sensor 28 where both the rotational sensor 46 and the cartridge sensor 28 send a recorded data reading to the micro processor 56 for the micro processor to convert the recorded data reading into numerical amounts 51 of the dosage 50. The display 52 shows the numerical amount 51 of the dosage 50 of the medicine 20 in the cartridge 16 based on the readings from the rotational sensor 46 and from the cartridge sensor 28.

A battery 58 is positioned within the interior of the dispenser 12. The battery is positioned adjacent relative to the micro processor 56. The battery 58 is disposable and can be changed when the power within the battery is fully diminished. The battery 58 sends electric power to the rotational sensor 46 and the cartridge sensor 28.

Furthermore, the battery sends power to the micro processor 56 so the micro processor can convert the recorded data reading of the rotational sensor 46 and the cartridge sensor 28 into the numerical amount 51 of the dosage 52. The battery 58 sends electrical power to the display 52 whereby the display is able to emit the numerical amount 51 of the dosage 50.

An injection button 60 is coupled to the distal end 40 of the dispenser 12 relative to the injection port 32. The injection button 60 protrudes out from the dispenser 12 and is coupled to a thumb tab 62. The injection button 60 protrudes out for a thumb of the user 64 to engage with the injection button 64 by pushing in the injection button 64 towards the dispenser 12. The injection button 60 protruding out of the dispenser 12 is configured for providing a feasible arrangement for the thumb of the user 64 to engage with the injection button 64.

When the injection button 60 is pushed into the dispenser 12, the rubber plunger head 48 rapidly protrudes forward within the cartridge 16 to expel the medicine 20. The thumb of the user 64 is configured for being positioned on a top surface 63 of the thumb tab 62 when the dosage ring 42 is adjusting the dosage 50 of the medicine 20 within the cartridge 16.

In use, an inlet cap 68 is engaged by threading to the injection port 32. The user twists the dosage ring 42, and the rubber plunger head 48 retracts. The medicine 20 is suctioned into the cartridge 16. The dosage 50 of the medicine 20 within the cartridge 16 is measured by the measuring scale 22 on the side and by the rotational sensor 46 within the dispenser 12. The rotational sensor 46 sends its readings to the micro processor 56 through a plurality of wiring 66. The micro processor 56 displays the numerical amount 51 of the dosage 50 on the display 52.

When a dosage 50 is ready to be injected, an outlet cap 70 is engaged by threading to the injection port 32. The outlet cap 70 is positioned to a destination that the medicine will be distributed to. The thumb of the user 64 resting on the thumb tab 62 of injection button 60 then pushes down towards the dispenser 12 wherein the rubber plunger head 48 pushes the medicine 20 out of the cartridge 16. The cartridge sensor 28 sends its readings to the micro processor 56 through a plurality of wiring 66. The micro processor 56 sends the numerical amount 51 of the dosage 50 within the cartridge 16 to the display 52.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medicine dosage control injecting device comprising:
a dispenser, said dispenser containing a cartridge, said cartridge storing a medicine, said dispenser having a rotational sensor and a cartridge sensor;
a dosage ring, said dosage ring configured for laterally rotating to adjust a dosage within said cartridge
a display, said display configured for showing numerical amount of said dosage within said cartridge from a value provided by said rotational sensor and said cartridge sensor; and
an injection button, said injection button being configured for a force to push in said injection button towards said dispenser wherein said cartridge releases said medicine.

2. The medicine dosage control injecting device of claim 1, further comprising said dispenser having a pen shaped body.

3. The medicine dosage control injecting device of claim 1, further comprising said medicine being a liquid.

4. The medicine dosage control injecting device of claim 1, further comprising said dispenser having an injection port, said injection port being an inlet and an outlet for said liquid medicine from said cartridge, said injection port being internally threaded, said injection port having a variety of caps to be engaged by threading to said injection port.

5. The medicine dosage control injecting device of claim 1, further comprising said cartridge being transparent and having a measuring scale.

6. The medicine dosage control injecting device of claim 1, further comprising said rotational sensor and said cartridge sensor being in electric communication with a micro processor.

7. The medicine dosage control injecting device of claim 1, further comprising said dispenser having a battery, said battery in electric communication with said micro processor.

8. The medicine dosage control injecting device of claim 1, further comprising said dosage ring coupled to a distal end of said dispenser relative to said injection port, said dosage ring being in electric communication with a rotational sensor.

9. The medicine dosage control injecting device of claim 1, further comprising said dosage ring being mechanically coupled to a rubber plunger head, said rubber plunger head being positioned within said cartridge, said rubber plunger head configured for adjusting said liquid medicine within said cartridge to relative amount of said dosage set by said dosage ring.

10. The medicine dosage control injecting device of claim 1, further comprising said display being a light emitting diode screen, said display being in electric communication with said micro processor.

11. The medicine dosage control injecting device of claim 1, further comprising said injection button being coupled to said distal end of said dispenser relative to said injection port, said injection button protruding out from said distal end of said dispenser, said injection button being in communication with said cartridge.

12. A medicine dosage control injecting device comprising:
a dispenser, said dispenser having a pen shaped body, said dispenser containing a cartridge, said cartridge storing a medicine, said medicine being a liquid, said dispenser having an injection port, said injection port being an inlet and an outlet for said liquid medicine from said cartridge, said injection port being internally threaded, said injection port having a variety of caps to be engaged by threading to said injection port, said cartridge being transparent and having a measuring scale, said dispenser having a rotational sensor and a cartridge sensor, said rotational sensor and said cartridge sensor being in electric communication with a micro processor, said dispenser having a battery, said battery in electric communication with said micro processor;
a dosage ring, said dosage ring coupled to a distal end of said dispenser relative to said injection port, said dosage ring being in electric communication with a rotational sensor, said dosage ring configured for laterally rotating to adjust a dosage within said cartridge, said dosage ring being mechanically coupled to a rubber plunger head, said rubber plunger head being positioned within said cartridge, said rubber plunger head configured for adjusting said liquid medicine within said cartridge to relative amount of said dosage set by said dosage ring;
a display, said display being a light emitting diode screen, said display being in electric communication with said micro processor, said display configured for showing numerical amount of said dosage within said cartridge from a value provided by said rotational sensor and said cartridge sensor; and
an injection button, said injection button being coupled to said distal end of said dispenser relative to said injection port, said injection button protruding out from said distal end of said dispenser, said injection button being in communication with said cartridge, said injection button being configured for a force to push in said injection button towards said dispenser wherein said cartridge releases said medicine through said injection port.
